(19) United States
(12) Patent Application Publication
(10) Pub. No.: US 2016/0004315 A1
(43) Pub. Date: Jan. 7, 2016

(54) SYSTEM AND METHOD OF TOUCH-FREE OPERATION OF A PICTURE ARCHIVING AND COMMUNICATION SYSTEM

(71) Applicant: PACSPoint Inc., Charlottesville, VA (US)

(72) Inventors: Jose Morey, Charlottesville, VA (US); Peter Stoll, Lansdale, PA (US)

(21) Appl. No.: 14/323,266

(22) Filed: Jul. 3, 2014

(51) Int. Cl. G06F 3/01; G06F 19/00; A61M 25/01; A61B 19/00; A61B 17/34; G06F 3/0484; A61B 5/06

(52) U.S. Cl. CPC G06F 3/017; G06F 3/0484; G06F 19/321; A61B 5/066; A61B 19/5244; A61B 17/3403; A61M 25/0105; A61B 2019/5236; A61B 2019/524; A61B 2019/5276; A61B 2019/5263; A61B 2019/5255; A61B 2017/00207

(57) ABSTRACT

A method of controlling a PACS image viewer using a system comprising one or more infrared cameras and a processor is described. The method comprises accepting user input from the one or more infrared cameras comprising hand motion, vector, and gesture information, sending the hand motion, vector, and gesture information to the processor according to a frame rate, and translating the hand motion, vector, and gesture information into a virtual input by the processor according to a set of computer-executable instructions, wherein the virtual input is configured to control a PACS image viewer. The virtual input simulates one or more key strokes, mouse clicks, or cursor movements and allows a physician to use a PACS image viewer without using any hand-operated equipment such as a mouse, trackpad, or keyboard. In this way, the physician may scroll through images using the PACS image viewer while maintaining a sterile environment.

### BACKGROUND OF THE INVENTION

1. Field of the Invention

The present disclosure relates to systems and methods of computer-implemented touch-free interfacing with a Picture Archiving and Communication System (PACS) image viewing software. Embodiments of the present disclosure relate to systems and methods that use a sensor configured for detecting hand gestures in combination with software for translating the hand gestures into specific controls for controlling a PACS image viewer.

2. Description of Related Art

In healthcare environments, it is very important to maintain sterility while interacting with patients especially during surgical procedures. In many cases, it is necessary to be able to use PACS while maintaining sterility. Presently, this is achieved in one of two ways. The physician can physically interact with a computer on which PACS is stored through direct contact with a mouse and/or keyboard, then resterilize. Alternatively, the physician can indirectly use PACS by instructing an assistant to physically interact with the computer and control PACS according to the physician's needs. Both of these methods are inefficient in their use of time and human resources.

There exist numerous ways of interacting with computers with contact-free methods, some of which involve medical settings. However, many of these involve interacting with operating system-level functions, like cursor movement and mouse clicks. While it is possible to use a computer in this manner, it is neither easy nor efficient, as the accuracy of current motion-tracking is not sufficient for the fine control needed to interact with GUIs intended for mouse control.

For example, U.S. Pat. No. 7,698,002 B2 describes a gesture-based way of controlling a medical device, specifically a pulse oximeter. It compares any received gestures with gestures in its database, then executes the commands associated with the appropriate gesture from its database. International Patent Application Publication No. WO 2013035001 A2 uses a gesture-based interface to control a group of medical devices. Gesture tracking uses markers on the user. U.S. Patent Application Publication No. 20110304650 A1 describes a generic user interface which receives motion data from a gesture-based controller, recognizing screen coordinates as extensions of objects pointed at it, while International Patent Application Publication No. WO 2007137093 A2 describes contact-free interaction with a computer for medical purposes. It uses an instrument with a tracking device that is visible to a motion-based controller and foot pedals that map to mouse clicks. In this way, the operator is able to control a cursor on a computer without physically coming into contact with the computer. However, despite these attempts, there remains a need for a touch-free method and system of allowing a physician to access medical records while maintaining a sterile environment.

### SUMMARY OF THE INVENTION

According to embodiments of the invention, software is provided in the form of a computer-readable storage device comprising computer-executable instructions configured to direct a processing module to: (a) receive hand position, vector, or gesture information from a sensor according to a specified frame rate; (b) translate the hand position, vector, or gesture information into one or more key strokes, mouse clicks, or cursor movements; and (c) navigate, annotate, or modify one or more images in a picture archiving and communication system (PACS) by instructing the system that the one or more key strokes, mouse clicks, or cursor movements have been performed. It is understood that in the context of this invention the software provided in the form of a computer-readable storage device can be presented to users as a Software As a Service (SAS) type product where the software is stored in one location and accessed remotely by users over the internet.

Such software can comprise computer-executable instructions configured to direct a processing module to: (a) compare one or more if statements that check for specific hand gesture data with a frame comprising hand gesture data to determine if a condition is met; and (b) if the condition is met, generate the instructions to navigate, annotate, or modify the one or more images, wherein the if statements are provided in a first listener class and the frame is provided in a second controller class.

In embodiments, the computer-readable storage devices can comprise a second controller class configured to comprise one or more frames comprising hand gesture data received from one or more infrared cameras, and a first listener class configured to receive information from the second controller class and execute the navigating, annotating, or modifying actions based on information received.

In preferred embodiments, the computer-readable storage devices can comprise computer-executable instructions configured to direct a processing module to apply an algorithm to x and y coordinates of the hand position to determine cursor location and movement, which algorithm calculates a cursor speed proportional to a position vector.

The present disclosure also provides methods of controlling a PACS image viewer using a gesture-based user interface. Such user interface includes a computer paired with a camera-based motion sensor, capable of tracking one or more of user hands, fingers, other tools, and gestures without the user being required to wear or come into contact with any special equipment. This motion sensor is supported by an API (application programming interface) that is used to provide data about the objects in its field of detection, such as the type of objects, their orientation and position, etc. It is also able to report data about types of gestures, with specific parameters relevant to each gesture (whether it is a swipe, screentap, keytap, etc.).

Such user interface also includes software that interfaces with the aforementioned API. This software is able to use object and gesture data from the motion sensor to algorithmically recognize supplementary gestures in real time, as they are signaled by the user. All types of gestures recognized by this software, as well as their parameters (speed, location, etc.), if necessary, are then processed by an event-based algorithm. This subroutine is called when the corresponding gesture occurs. When called, the algorithm is capable of interpreting the gesture and parameter and sending some number of signals to the PACS image viewer, in the form of simulated keystrokes, mouse clicks, and/or cursor movements. In this manner, touch-free navigation and control of the PACS image viewer is possible, i.e., a physician can control the computer on which PACS resides to navigate through and/or manipulate PACS-stored images without physically touching the computer.

By interacting in a contact-free manner with the computer on which PACS is running, the physician can use PACS to access desired images without compromising sterility during a medical procedure. This eliminates both the need for spending extra time maintaining sterility and the need for an assistant.

In embodiments, provided are methods of controlling a picture archiving and communication system (PACS), the method comprising: (a) receiving hand position, vector, or gesture information from a sensor optionally according to a specified frame rate; (b) translating the hand position, vector, or gesture information into one or more key strokes, mouse clicks, or cursor movements; and (c) navigating, annotating, or modifying one or more images in a picture archiving and communication system (PACS) by instructing the system that the one or more key strokes, mouse clicks, or cursor movements have been performed.

Such methods can comprise (d) comparing one or more if statements that check for specific hand gesture data with a frame comprising hand gesture data to determine if a condition is met; and [symbol] if the condition is met, generating the instructions to navigate, annotate, or modify the one or more images, wherein the if statements are provided in a first listener class and the frame is provided in a second controller class.

In embodiments, the second controller class can be configured to comprise one or more frames comprising hand gesture data received from one or more infrared cameras, and the first listener class can be configured to receive information from the second controller class and execute the navigating, annotating, or modifying actions based on information received.

Such methods can comprise applying an algorithm to x and y coordinates of the hand position to determine cursor location and movement, which algorithm calculates a cursor speed proportional to a position vector.

For example, provided is a method of controlling a PACS image viewer using a system comprising one or more infrared cameras for detecting certain hand gestures and a processor for translating hand gestures into controls for operating the computer on which PACS resides. Such a method can comprise: i) accepting user input from one or more cameras (preferably infrared cameras), wherein the user input is one or more of hand motion, vector, and/or gesture information, ii) sending the hand motion, vector, and/or gesture information to a processor according to a specified frame rate, iii) having the processor translate the hand motion, vector, and/or gesture information into a virtual input according to a set of computer-executable instructions, wherein the virtual input is configured to control one or more functions available in a PACS image viewer system.

In embodiments, the hand gestures serve as a virtual input to simulate one or more key strokes, mouse clicks, or cursor movements. The one or more simulated key strokes, mouse clicks, or cursor movements are configured to allow a physician to select particular images, select particular series, change series, scroll through image stacks, scroll through series stacks, move a cursor, window and level an image, annotate images, and bring up a tool bar while maintaining a sterile environment.

According to embodiments of this disclosure, a physician can perform a number of functions for navigating a PACS system of images while maintaining a sterile environment by not physically interacting with a computer. Voice recognition technology can also be used to control the computer on which the PACS images are viewed, as well as forgoing use of a mouse, trackpad, or keyboard. These features can be used instead of interacting indirectly with a computer to access patient-related images in PACS, such as by way of an assistant. Using systems and methods of the invention a healthcare worker, such as a physician or surgeon, can perform any number of sterile procedures such as image-guided biopsies, image-guided drain placement, diagnostic imaging interpretation and manipulation/marking, imaging education, image-guided surgical intervention, and image guided endovascular intervention. With aid of the invention, these sterile procedures can be conducted by the physician without the physician physically interacting with or touching a computer on which the images of the patient's body reside. These touch-free, sterile procedures will be elaborated on in the foregoing Examples.

Also included within the scope of the invention are systems for controlling a picture archiving and communication system (PACS), the system comprising: (a) a PACS system in operable communication with a sensor for identifying hand movements and gestures of a user; and (b) software for translating the one or more hand gestures into one or more key strokes, mouse clicks, or cursor movements for controlling the PACS system.

Such systems can further comprise one or more imaging modality in operable communication with the PACS system, such as one or more of MRI (magnetic resonance imaging), PET (positron emission tomography), CT (computed tomography), X-ray, Ultrasound, Photoacoustic imaging, Fluoroscopy, Echocardiography, or SPECT (single-photon emission computed tomography).

### BRIEF DESCRIPTION OF THE DRAWINGS

The accompanying drawings illustrate certain aspects of embodiments of the present invention, and should not be used to limit the invention. Together with the written description the drawings serve to explain certain principles of the invention.

FIG. 1 is a schematic diagram of a system embodiment of this disclosure.

FIG. 2 is a schematic diagram of a method embodiment of this disclosure.

FIGS. 3A and 3B are schematic diagrams of a method embodiment of this disclosure showing control of a PACS system using a LEAP controller.

### DETAILED DESCRIPTION OF VARIOUS EMBODIMENTS OF THE INVENTION

Reference will now be made in detail to various exemplary embodiments of the invention. It is to be understood that the following discussion of exemplary embodiments is not intended as a limitation on the invention. Rather, the following discussion is provided to give the reader a more detailed understanding of certain aspects and features of the invention.

One embodiment of a system of this disclosure uses one or more motion sensing input devices to detect hand gestures. The motion sensing input devices may comprise one or more infrared (IR) cameras. For example, a LEAP Motion controller may be used. The LEAP Motion controller is a small USB device containing two upwards-facing stereoscopic IR cameras, which establish an inverted pyramid-shaped detection region of height approximately equal to 2 feet above the device. The software controlling the device has an API (application programming interface) with support for up to six programming languages. The API v1 contains support for finger tracking as well as gesture tracking. A new API v2 has recently been released, providing tracking for individual joints. However, other motion sensing input devices could also be used or modified as needed, such as KINECT (MICROSOFT), PLAYSTATION Eye, NINTENDO Wii or ASUS Xtion PRO. The system typically comprises one or more remote sensing input devices connected to a processor. The processor is further connected to a display and has access to a memory comprising a set of computer-executable instructions for performing algorithms which translate vectors and gestures into cursor movement, mouse clicks, and keystrokes. An exemplary algorithm would observe the position of the index fingertip with respect to the LEAP Motion controller's detection plane along the x and y axes and move the cursor to the corresponding location on the display. It would also observe any programmed gestures, such as screen taps and key taps, which the LEAP software handles, custom gestures, or even user-defined gestures, and carry out an action based on the mapping of these gestures to PACS functions.

The computer-executable instructions may be set up as a two-class program that contains a Listener Class and a Controller class. The Listener class is customized to receive the information from the controller class and then act on the information received. The design of the Listener class was done with blocks of if statements checking for various conditions in the information received from the controller class, and then modifying the results of that input as needed. The computer-executable instructions may report coordinates of objects in the field of view of the motion sensing input device using the x, y, and z axis with the middle of the input device representing the origin point. The computer-executable instructions use the x and y coordinates delivered by the input device and use an algorithm to determine where the cursor should move on a computer screen. The computer-executable instructions also allow for configuration of a group of hand gestures that can be used as additional inputs. These gestures have been configured to allow a user to operate PACS software using a combination of the gestures available.

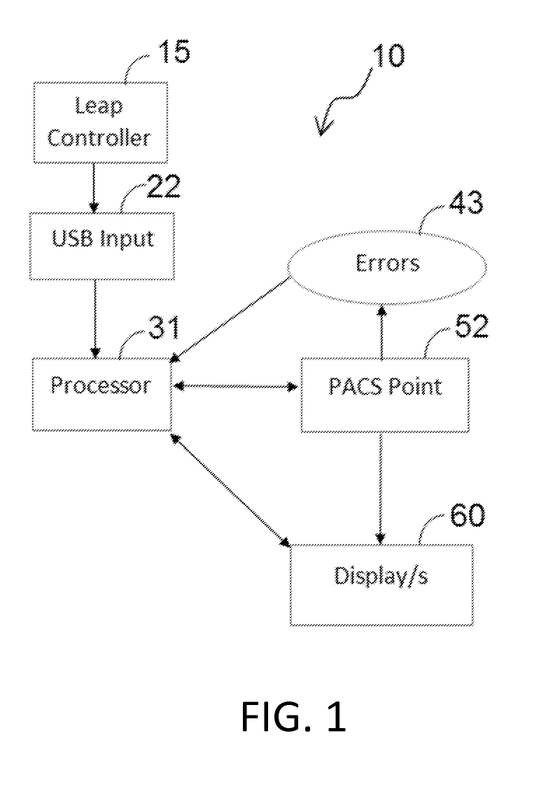

Turning now to the figures, FIG. 1 shows a specific embodiment of such a system 10 where a motion sensing input device, which in this case is a LEAP Motion controller 15 is connected to a computer via USB interface 22. This USB interface 22 is accessible to a processor 31, which receives data from the interface 22, display(s) 60, PACS Point program 52 (also referred to herein as a set of computer-executable or computer-readable instructions which may be used interchangeably with “PACS point program” or “PACS point”), and errors 43 generated by the PACS Point program. PACS Point program 52 consists of computer-readable instructions that can be executed by the processor 31, which affect operations at the OS level, as well as the display 60. The computer-readable instructions can be programmed in any suitable programming language, including JavaScript, C#, C++, Java, Python, and Objective C.

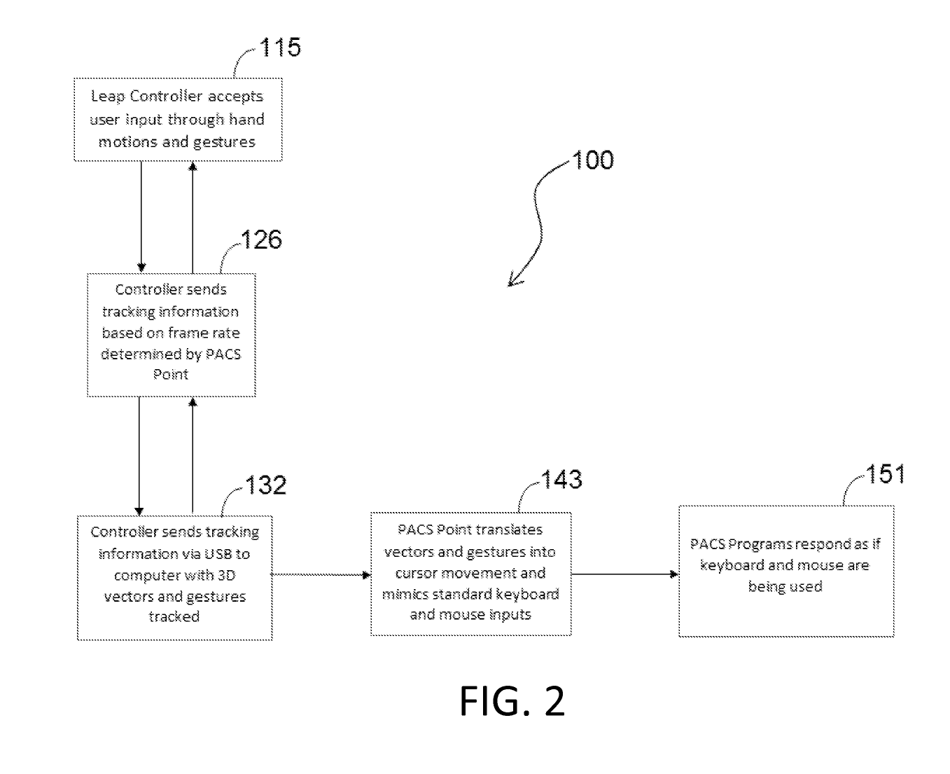

FIG. 2 shows software-level operations 100 of an embodiment of the disclosure. The LEAP Motion controller accepts user input through hand motions and gestures 115. Associated data is sent via USB to a computer as 3D vectors and gestures 132. Example schemes of this tracking include an index-to-thumb pinch for zooming, two hands moving toward/away from each other for zooming, five fingers spread out moving up or down for scrolling, one finger screen tap click for mouse click, two finger screen tap click for mouse click, three finger screen tap click for mouse click and hold, one hand grab for mouse click and hold, one finger keytap for right mouse click, one finger or thumb circle for scrolling (clockwise or counterclockwise), one hand swipe left or right for keyboard input for next series, and one finger movement for cursor movement. This tracking information is based on a frame rate determined by PACS Point 126. Typically, the highest frame rate possible for a particular component of the system is preferable. PACS Point translates vectors and gestures into cursor movement, mouse clicks, keystrokes, etc., which are emulated at the OS level 143. PACS programs then respond as if keyboard and mouse are used 151.

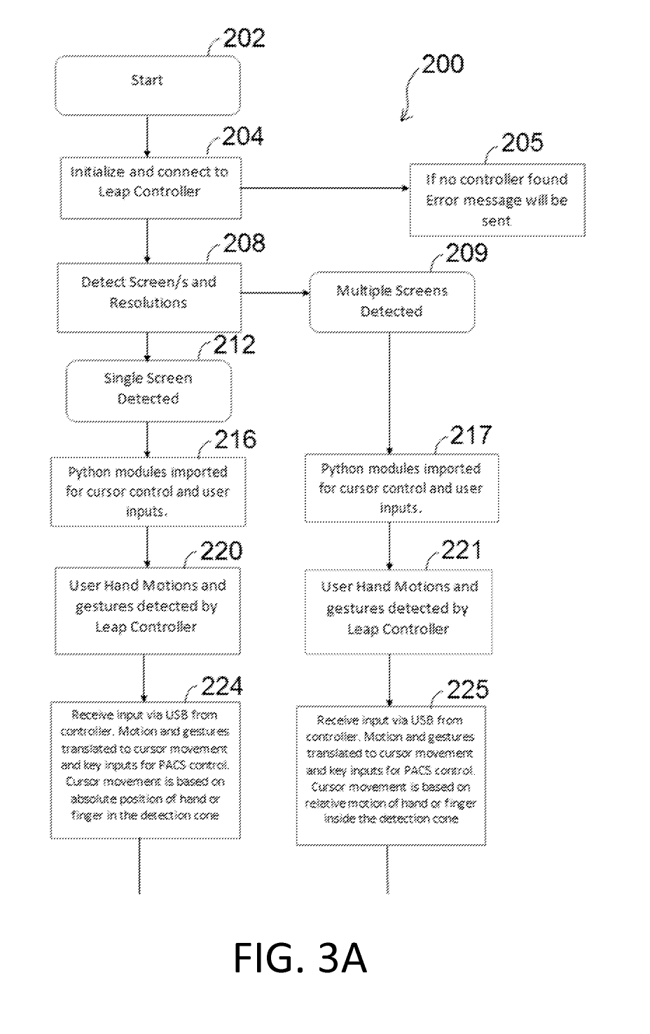

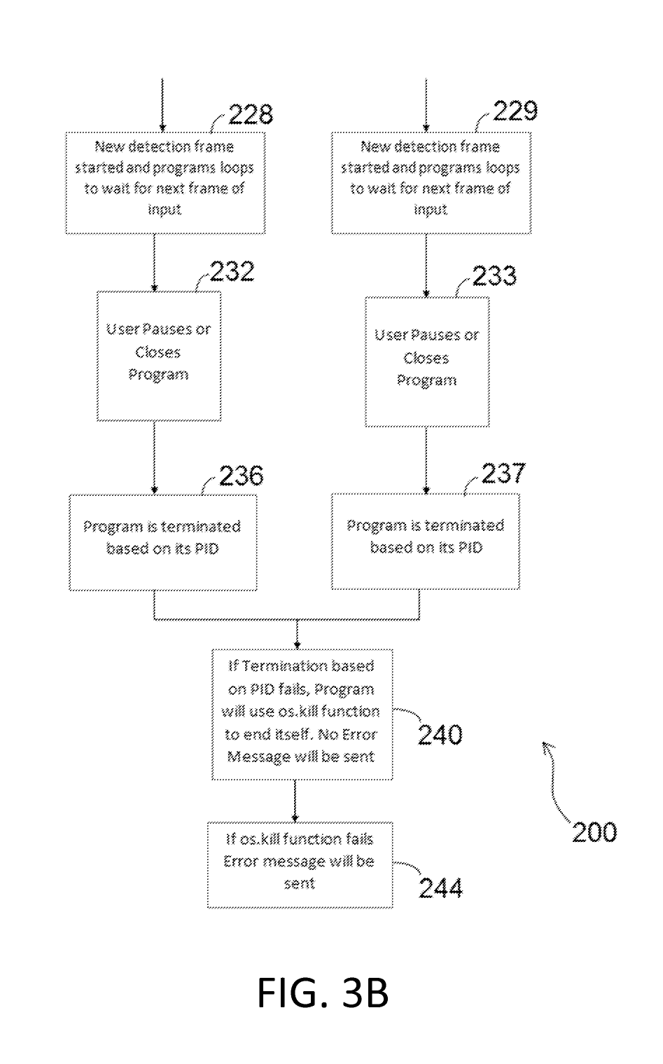

FIGS. 3A and 3B show control flow 200 of an embodiment of a method of this disclosure which may be embodied in a computer program or application. The control flow begins at reference numeral 202. The program is initialized and a connection to the LEAP Motion controller is made 204. If no valid controller is found, an error message is sent 205. The controller is configured by the program, and the program is enabled to receive events from the controller. The number of screen(s) is detected 208, which is important in the calculation of cursor movement because as multiple displays are connected, the display dimensions become less similar to the LEAP Controller's detection area dimensions, so an alternate cursor control scheme should be used to ensure full coverage of both displays. The control flow then may be executed in two branches, depending on whether a single screen 212 or multiple screens 209 are detected; even reference numerals represent the branch where a single screen is detected, and odd reference numerals represent the branch where multiple screens are detected. Necessary program instructions are imported to allow OS level control over cursor movement, mouse clicks, keystrokes, etc. 216, 217.

The program instruction(s) are imported according to the following lists, wherein step 216 imports the instruction(s) from List 1, and step 217 imports the instruction(s) from List 2.

| List 1 | List 2 |
|---|---|
| LEAP | LEAP |
| Autopy | Win32api |
| Win32api | Win32con |
| Win32con | Os |
| Os | Sys |
| Sys | Wxpython |
| Wxpython | Pygame |
| Pygame | PIL |
| PIL | |

Win32con is used to provide the key constants used by Win32api to mimic keyboard events. Win32api (also called PyWin) is used to contain the program in a “wrapper”, which allows the program to run as a service on Windows. This module is also involved in the installation of the program to any Windows computer that may use it. AutoPy is used for a variety of purposes, such as allowing the program to detect the size of the monitor that the program will be running on in order to adjust the algorithm used in positioning the cursor on the screen. Additionally, AutoPy is linked to the recognized LEAP gestures and allows the program to simulate keyboard or mouse inputs that are used to interact with PACS imaging applications. Sys provides system-specific parameters and functions and Os provides portable way of using operating system dependent functionality. WxPython is a GUI toolkit for the Python programming language. Pygame is a cross-platform set of Python modules designed for writing video games. It includes computer graphics and sound libraries designed to be used with the Python programming language. Python Imaging Library (PIL) adds image processing capabilities to a Python interpreter.

In a loop, user hand motions and gestures are detected by the LEAP Motion controller 220, 221, and data is transmitted via USB to the computer 224, 225. Motion and gestures are translated to cursor movement, mouse clicks, and keystrokes for PACS control 224, 225. If the system is displayed on a single screen, cursor movement is based on the absolute position of the hand and/or finger in the detection area 224. If the system is displayed on multiple screens, cursor movement is based on the relative motion of the hand and/or finger in the detection area 225. A new detection frame is started, and the program loops to wait for next frame of input 228, 229. The loop is broken when the user pauses or closes the program 232, 233. The program is terminated based on its PID at the OS level 236, 237. At this point, both branches converge to the same termination steps. If termination based on PID fails, the program will attempt to force quit 240. If this fails, an error message is sent 244.

In specific embodiments of the invention methods of using the software or methods of controlling the PACS system can be used to perform medical procedures in real time. A physician can perform a procedure on a patient while accessing images of the patient's body during the procedure itself, yet while maintaining sterility of the environment in which the patient is undergoing the procedure. For example, a method of guiding a needle or catheter within a patient's body, can comprise: (a) imaging a region of interest of a patient's body with one or more imaging modality to obtain one or more images of the region of interest; (b) accessing the one or more images in a picture archiving and communication system (PACS); (c) navigating the one or more images in the PACS system to locate an image or images showing a position of a needle or a catheter within the patient's body, wherein the navigating is performed using a computer hardware sensor device that supports hand and finger motions as input; and (d) moving the needle or catheter to another position within the patient's body using the one or more images in the PACS system as a guide to desired positioning.

Such methods can comprise navigating of the one or more images which involves one or more of selecting one or more images, selecting a series of images, changing image series, scrolling through image stacks, scrolling through image series stacks, moving a cursor, annotating an image or images, or accessing a tool bar. The navigating is preferably performed while maintaining a sterile environment for the patient, such as by having no physical interaction with a computer. Such navigating of the PACS images is preferably performed directly by the physician intraoperatively.

In preferred embodiments, the physician or healthcare worker can annotate images electronically and without physically interacting with a computer.

The one or more imaging modality can be chosen from one or more of MRI (magnetic resonance imaging), PET (positron emission tomography), CT (computed tomography), X-ray, Ultrasound, Photoacoustic imaging, Fluoroscopy, Echocardiography, or SPECT (single-photon emission computed tomography).

These methods, as well as other specific methods of using the software and image navigating methods of the invention are explained in more detail in the Examples provided below.

### Example 1

The LEAP Motion Controller is designed to recognize hands, fingers, and gestures within its detection cone and to report that information in the form of 3D coordinates and gesture tracking. The LEAP Motion sends this information to the computer via USB. PACS Point works with the LEAP Motion Controller and uses this tracking information to produce commonly used inputs, such as cursor movement and mouse clicks, for medical image viewing programs such as GE PACS. PACS Point is coded in Python v 2.7 and uses several standard Python modules as well as the Python module generated for use with the LEAP Controller. When PACS Point starts it first checks to make sure a LEAP Controller is connected and then determines the size of the display screen. When the controller is found the program sets policy flags that ensure that PACS Point can run in the background when it does not have the computers focus. The LEAP Controller sends information on a frame by frame basis, that can have varying speeds depending on the settings of the controller and the environment it is in. PACS Point calls the on_frame method supplied by the LEAP Python module to receive this information in a continuous loop until the program is closed. The general structure of PACS Point is set up as a two-class program that contains the Listener Class and the standard controller class supplied by LEAP. The Listener class is customized to receive the information from the controller class and then act on the information received. The design of the Listener class was done with blocks of if statements checking for various conditions in the information received from the controller, and then modifying the results of that input as needed.

The Listener class will first check for the number of fingers within the controller's detection cone. This number will determine which if statement block the rest of the incoming information should be read by. For example if the incoming information shows that there are 5 fingers within the detection cone the Listener class will then look at the direction and the position of the palm of that hand. Depending on the direction and the position of the palm the Listener class will then generate a virtual keystroke that is recognized by the operating system (i.e. Windows) and is treated the same way as a physical keystroke would be. Depending on the imaging program that is being run with PACS Point, if PACS Point detects 5 fingers in the detection cone it will generate the keystroke needed to control screen scrolling in the desired direction. Controlling the cursor with PACS Point is done by moving one finger into the detection cone of the controller. The if statement block for one finger will take the screen size determined upon initialization of the program and acquire the 2-D position of the finger-tip position. Using the LEAP API's built in translation probability attribute as a filter to reduce stuttering, PACS Point will send the 2-D position values to an algorithm based on screen size to produce coordinates for the mouse to move to. This can be done by either moving the cursor to the position on the display corresponding to the 2-D position detected by the LEAP controller or by moving the cursor at a velocity proportional to a position vector of this 2-D position. If a finger-tip is detected in the top right corner of the controller's detection cone, the cursor will move to the top right corner of the display screen. These coordinate values are constantly updated by the frames of data the LEAP Controller sends, which allows for smooth cursor movement during use.

The LEAP Controller is capable of recognizing several gestures including, what are known as keytaps, screentaps, swipes, and circles. When a gesture is recognized by the LEAP Controller it is reported to PACS Point with the gesture type and the gesture progress on a frame by frame basis. PACS Point's gesture system is designed to work when 1, 2, or 3 fingers are detected in the controller's cone. If PACS Point determines that there are 2 fingers in the cone, the cursor will stop moving and the program will wait to see if a gesture is made. If the user taps their fingers towards the screen, PACS Point will register that gesture as a screen tap, and will simulate a Left-button mouse click, which is recognized by the operating system as equivalent to a physical Left-button mouse click. If 3 fingers are detected and the same motion is repeated, PACS Point will simulate a Left-mouse button hold that is useful for drag-and-drop operations. If only 1 finger is detected, PACS Point will not look for the screen tap gesture, but will instead look for a keytap which is a downward motion similar to typing on a keyboard. If a keytap is registered, PACS Point will simulate the keystroke needed to bring up the toolbar, depending on the imaging program that it is being run with (i.e. Right-button mouse click with GE PACS). Errors are handled at the end of the customized on_frame class method and will generally tell PACS Point to go back to the last good frame of data and wait until another good frame of data is available. This system of error handling is also designed to reduce cursor stuttering. When the user decides to close PACS Point, all processes created by the program are identified by their PID and terminated using the standard sys module of Python. If this fails for any reason the os.kill function is called which will kill the PACS Point process and child processes it created. This is done in order to make sure that no left over virtual keystrokes are being read by the operating system.

### Example 2

### Image-Guided Biopsies

The software may allow the physician to directly navigate and/or manipulate images in PACS using the controller connected to a scanner or associated PACS computer. The physician will be able to select particular images, select particular series, change series, scroll through image stacks, scroll through series stacks, move the cursor, window and level the image, annotate the images, bring up the tool bar, adjust and change the PACS preferences, refresh screen, minimize the PACS screen and close PACS all without using a mouse or keyboard. This would allow the physician to maintain sterility throughout the procedure. The physician would use the service to guide the needle through the patient's body or tissue to the correct position without using a mouse/keyboard to navigate to the relevant image or without having to direct an assistant to the right image. The needle position will be able to seen on the images selected by the touch free technology. The physician will be able to scroll through as many needed images as required to find the needle position to accurately pinpoint its location without the use of a mouse or keyboard, thereby maintaining sterility throughout the entire procedure. The inventors have found that for a user, such as a physician, to localize 10 individual images by directing a technologist averaged 3.75 minutes. A physician using the touch-free device localizing the same 10 images averaged 1.50 minutes, which represents a decrease in image localization by over 50%.

### Example 3

### Image-Guided Drain Placement

The software may allow the physician to directly manipulate the images via the controller connected to an MRI/CT scanner or associated PACS computer. This will allow the physician to retain sterility throughout the procedure. The physician will use the service to guide the drainage catheter to the correction position without the need of a mouse/keyboard and without having to direct an assistant to the right image. The images will be able to be scrolled through to find the needle position by using a palm open gesture over the LEAP controller. The hand will be moved up to scroll upwards through the images and moved down to scroll down through the images. The cursor will be moved by extending the index finger over the LEAP controller and moving it through the air. The tool box will be brought up by tap downwards in the air with the index finger. Any tool or image in the toolbar may be selected by extending both the index finger and thumb and simulating a push forward over the controller. Image rotation, refreshing screen, and image inversion can then be selected by moving the index finger through the air until the cursor is placed over the appropriate function button. The button is then selected by extending both the index finger and thumb over the controller and pushing forward over the controller. Drag/drop functionality will be performed by extending three fingers over the controller and pushing forward. This will allow a user to initiate PACS functions such as length measurements and region of interest analysis if radiologic Hounsfield unit measurements are required. This is currently implemented and has decreased image localization by over 50%.

### Example 4

### Touch-Free Image Manipulation/Marking

The service will allow physicians to manipulate any PACS system and aid in image interpretation. This will allow the physician to not only scroll through any series and image number but to manipulate and mark the DICOM data without physically touching a mouse or keyboard. DICOM (digital imaging and communications in medicine) data is a standard for storing and transmitting information in medical imaging and enables the integration of various types of scanners into PACS. Marking is performed by opening the toolbox, which is performed by pushing the index finger downward over the LEAP controller. Any tool can then be selected by extending the index finger and thumb and pushing forward over the controller. The initiation of ruler, annotation arrow, text box, magnification tool, window tool, and region of interest or level tool is initiated by extending 3 fingers over the controller and pushing forward.

### Example 5

### Touch-Free Imaging Education

The service will allow a healthcare professional to manipulate any DICOM imaging for the purpose of education touch free. This would allow the training professional to remain in a sterile environment. This will also allow any trainees to manipulate the imaging data and also remain sterile throughout the process.

### Example 6

### Touch-Free Image-Guided Surgical Intervention

The service will allow the physician to manipulate images intraoperatively and not only be able to remain sterile but not rely on an assistant to manipulate the images. In most current practices, the physician has to verbally direct the technologists to scroll through images, draw arrows, draw regions of interest, change series, change image and draw length measurements so that the physician may maintain sterility throughout the procedure. The software will enable the physician to manipulate the images in any way as currently practiced by moving their hand over the LEAP controller to manipulate the images and remain sterile. This will increase patient safety by reducing the risk of infection, reducing anesthesia time and reduce cost by reducing operating room time.

### Example 7

### Touch-Free Image-Guided Endovascular Intervention

The software will allow the physician to directly manipulate the images by way of a controller connected to the scanner or associated PACS computer. This will allow the physician to retain sterility throughout the procedure. The physician will use the service to evaluate the fluoroscopic and digital images to aid in vascular anatomy interpretation, vascular pathology and endovascular intervention without the use of a mouse/keyboard and without having to direct an assistant to the right image.

The present invention has been described with reference to particular embodiments having various features. In light of the disclosure provided, it will be apparent to those skilled in the art that various modifications and variations can be made in the practice of the present invention without departing from the scope or spirit of the invention. One skilled in the art will recognize that the disclosed features may be used singularly, in any combination, or omitted based on the requirements and specifications of a given application or design. When an embodiment refers to “comprising” certain features, it is to be understood that the embodiments can alternatively “consist of” or “consist essentially of” any one or more of the features. Other embodiments of the invention will be apparent to those skilled in the art from consideration of the specification and practice of the invention.

It is noted that the singular forms “a,” “an,” and “the” include plural referents unless the context clearly dictates otherwise. It is intended that the specification and examples be considered as exemplary in nature and that variations that do not depart from the essence of the invention fall within the scope of the invention. Further, all of the references cited in this disclosure are each individually incorporated by reference herein in their entireties and as such are intended to provide an efficient way of supplementing the enabling disclosure of this invention as well as provide background detailing the level of ordinary skill in the art.

1. A computer-readable storage device comprising computer-executable instructions configured to direct a processing module to:
receive hand position, vector, or gesture information from a sensor according to a specified frame rate;
translate the hand position, vector, or gesture information into one or more key strokes, mouse clicks, or cursor movements; and
navigate, annotate, or modify one or more images in a picture archiving and communication system (PACS) by instructing the system that the one or more key strokes, mouse clicks, or cursor movements have been performed.

2. The computer-readable storage device of claim 1 comprising computer-executable instructions configured to direct a processing module to:
compare one or more if statements that check for specific hand gesture data with a frame comprising hand gesture data to determine if a condition is met; and
if the condition is met, generate the instructions to navigate, annotate, or modify the one or more images;
wherein the if statements are provided in a first listener class and the frame is provided in a second controller class.

3. The computer-readable storage device of claim 2, wherein the second controller class is configured to comprise one or more frames comprising hand gesture data received from one or more infrared cameras, and wherein the first listener class is configured to receive information from the second controller class and execute the navigating, annotating, or modifying actions based on information received.

4. The computer-readable storage device of claim 3 comprising computer-executable instructions configured to direct a processing module to apply an algorithm to x and y coordinates of the hand position to determine cursor location and movement, which algorithm calculates a cursor speed proportional to a position vector.

5. A method of controlling a picture archiving and communication system (PACS), the method comprising:
receiving hand position, vector, or gesture information from a sensor according to a specified frame rate;
translating the hand position, vector, or gesture information into one or more key strokes, mouse clicks, or cursor movements; and
navigating, annotating, or modifying one or more images in a picture archiving and communication system (PACS) by instructing the system that the one or more key strokes, mouse clicks, or cursor movements have been performed.

6. The method of claim 5 comprising:
comparing one or more if statements that check for specific hand gesture data with a frame comprising hand gesture data to determine if a condition is met; and
if the condition is met, generating the instructions to navigate, annotate, or modify the one or more images;
wherein the if statements are provided in a first listener class and the frame is provided in a second controller class.

7. The method of claim 6, wherein the second controller class is configured to comprise one or more frames comprising hand gesture data received from one or more infrared cameras, and wherein the first listener class is configured to receive information from the second controller class and execute the navigating, annotating, or modifying actions based on information received.

8. The method of claim 7 comprising applying an algorithm to x and y coordinates of the hand position to determine cursor location and movement, which algorithm calculates a cursor speed proportional to a position vector.

9. A method of guiding a needle or catheter within a patient's body, the method comprising:
imaging a region of interest of a patient's body with one or more imaging modality to obtain one or more images of the region of interest;
accessing the one or more images in a picture archiving and communication system (PACS);
navigating the one or more images in the PACS system to locate an image or images showing a position of a needle or a catheter within the patient's body, wherein the navigating is performed using a computer hardware sensor device that supports hand and finger motions as input; and
moving the needle or catheter to another position within the patient's body using the one or more images in the PACS system as a guide to desired positioning.

10. The method of claim 9, wherein the navigating of the one or more images involves one or more of selecting one or more images, selecting a series of images, changing image series, scrolling through image stacks, scrolling through image series stacks, moving a cursor, annotating an image or images, or accessing a tool bar.

11. The method of claim 9, wherein the navigating is performed while maintaining a sterile environment for the patient.

12. The method of claim 10, wherein the navigating is performed while maintaining a sterile environment for the patient.

13. The method of claim 11, wherein the sterile environment is maintained by having no physical interaction with a computer.

14. The method of claim 12, wherein the sterile environment is maintained by having no physical interaction with a computer.

15. The method of claim 10, wherein the annotating involves electronically marking the one or more images without physically interacting with a computer.

16. The method of claim 10, wherein the navigating of the one or more images is performed intraoperatively.

17. The method of claim 10, wherein the one or more imaging modality is chosen from one or more of MRI (magnetic resonance imaging), PET (positron emission tomography), CT (computed tomography), X-ray, Ultrasound, Photoacoustic imaging, Fluoroscopy, Echocardiography, or SPECT (single-photon emission computed tomography).

18. A system for controlling a picture archiving and communication system (PACS), the system comprising:
a PACS system in operable communication with a sensor for identifying hand movements and gestures of a user; and
software for translating the one or more hand gestures into one or more key strokes, mouse clicks, or cursor movements for controlling the PACS system.

19. The system of claim 18 further comprising one or more imaging modality in operable communication with the PACS system.

20. The system of claim 19, wherein the one or more imaging modality is chosen from one or more of MRI (magnetic resonance imaging), PET (positron emission tomography), CT (computed tomography), X-ray, Ultrasound, Photoacoustic imaging, Fluoroscopy, Echocardiography, or SPECT (single-photon emission computed tomography).
